(19) United States
(12) Patent Application Publication
(10) Pub. No.: US 2009/0136966 A1
(43) Pub. Date: May 28, 2009

(54) Normalization of Complex Analyte Mixtures

(75) Inventors: Laszlo Takacs, Newbury Park, CA (US); Andras Guttman, San Diego, CA (US); Mariana Kuras, Chatillon (FR)

Correspondence Address: NIXON & VANDERHYE, PC, 901 NORTH GLEBE ROAD, 11TH FLOOR, ARLINGTON, VA, 22203, US

(73) Assignee: BIOSYSTEMS INTERNATIONAL SAS, EVRY Cedex (FR)

(21) Appl. No.: 11/989,420

(22) PCT Filed: Jul. 27, 2006
(86) PCT No.: PCT/IB2006/003161

§ 371 Date: Jan. 25, 2008

Related U.S. Application Data
(60) Provisional application No. 60/702,860, filed on Jul. 28, 2005.
(60) Provisional application No. 60/781,001, filed on Mar. 11, 2006.

(51) Int. Cl. G01N 33/53; G01N 33/536; C40B 50/00; G01N 33/543

(52) U.S. Cl. 435/7.1; 436/536; 436/518; 506/23

(57) ABSTRACT

The present invention relates to methods and compositions for the normalization of complex analyte mixtures. The invention allows the preparation of profiled samples from highly complex analyte mixtures, allowing the identification of relevant targets or biomarkers. The invention also relates to methods for producing devices, such as a support, suitable for normalization of complex analyte samples. The invention can be used for the normalization of any complex mixture, such as immunogenic libraries, particularly of human source, and to identify or produce biomarkers highly relevant to human traits or conditions.

The present invention relates to methods and compositions for the normalization of complex analyte mixtures. The invention allows the preparation of profiled samples from highly complex analyte mixtures, allowing the identification of relevant targets or biomarkers. The invention also relates to methods for producing devices, such as a support, suitable for normalization of complex analyte samples. The invention can be used for the normalization of any complex mixture, such as immunogenic libraries, particularly of human source, and to identify or produce biomarkers highly relevant to human traits or conditions.

Small molecule metabolite, peptide and protein expression analysis is a growing field in the medicinal, veterinarian, food and environmental monitoring and profiling areas. In this respect, WO2005/077106 relates to a method of identifying biomarkers specific to a disease condition. A particularly important quasi random sampling based analytical method is mass spectrometry. While these methods are efficient, the inventors have now discovered that representational differences of individual analyte elements in a complex mixture inhibit comprehensive description via sampling based analysis. Accordingly, while presently existing methods allow the identification of biomarkers from complex analyte samples, a much more efficient approach can be designed by prior subjecting the complex analyte sample to a normalization step. In particular, normalization of representational differences enables mass spectrometry based methods aimed at global but qualitative analysis of the sample.

In particular, the invention is based on the observation, by the inventors, that polyclonal antibodies contain more and higher affinity antibodies against highly abundant elements of complex analyte mixtures used as immunogen for polyclonal antibody generation. Thus, allowing a complex analyte mixture to interact with the immobilized polyclonal antibody under equilibrium conditions will allow the formation of more antibody-antigen complexes of the highly abundant elements then the low abundance elements of the complex analyte mixture. Antibody-antigen complex formation kinetics is dependent on the antigen and antibody concentration and the affinity of antibodies. If the antibodies are immobilized on a saturated chromatography column or any other surface which allow antibody/antigen interaction; the complex formation kinetics will be dependent on the local antigen concentration and the interaction time, e.g. flow rate (in case the antigen is in the mobile phase).

Accordingly, as demonstrated by the inventors, sampling based analysis following presently available methods will repeatedly characterize elements that occur at higher abundance (concentration) levels, and there is a need to normalize representational differences. Normalization of representational differences will increase the likelihood of characterization of individual elements that are present at low abundance (concentration) levels. Processes that require a specific concentration level of an individual element in a complex mixture will not be initiated via all elements in the absence of normalization, while after normalization, more and possibly all elements could initiate processes that are concentration dependent.

The present invention thus relates to methods and devices for the normalization of complex analyte samples, as well as to the uses thereof, particularly to identify or produce biomarkers or biological targets.

The invention can be used, e.g., in the process of immunogen generation for monoclonal antibody library preparation. As a result of eliminated or reduced representational differences, each antigenic epitope present on any individual analyte will have similar chances to generate antibody response, while such likelihood presently mainly depends on antigenicity.

The invention can also be used e.g., in tracer preparation for screening individual antibodies or antibody arrays in “labeled tracer-cold inhibitor” type quantitative immunoassays. As a result of eliminated or reduced representational differences, each analyte element will generate relatively similar signal intensity when bound by a specific monoclonal antibody.

An object of this invention more specifically relates to a method of normalizing a complex analyte sample, the method comprising contacting said complex analyte sample with a binding composition comprising a polyclonal antibody generated against the complex analyte or a derivative thereof, under conditions that do not saturate the antigen-binding capacity of the binding composition, and recovering the sample that did not react with said binding composition, said sample being normalized.

In a particular embodiment, recovering the sample that did not react with the binding composition comprises removing all complexes formed.

The contacting step may be performed in solution. Alternatively, in a preferred embodiment, the binding composition is immobilized on a solid matrix, which may be selected from e.g., a bead, a stationary phase, a chromatography column, etc.

An important aspect of the invention is that the contacting is performed under conditions that do not saturate the binding capacity of the composition. Such conditions may be achieved e.g., by limiting the interaction time between the sample and the binding composition and/or by limiting the analyte concentration in the sample.

In a particular embodiment, the binding composition is immobilized on a column and the interaction time is limited by changing the flow rate of the column.

As will be disclosed below, the present invention demonstrates that changing the flow rate and/or the concentration of protein antigens loaded onto an affinity chromatography column, or any other surface which allow antibody/antigen interaction, prepared with saturating amounts of polyclonal antibody (generated against the same or partially identical complex protein analyte mixture, like the human serum, human plasma, depleted human serum or depleted human plasma) generates normalized protein mixes that contain partially overlapping populations of protein analytes. Any populations and the sum of the overlapping populations represent the analyte void of, or, at least with significant reduction of representational differences. The flow rate and/or concentration may be adjusted by the skilled artisan following conventional techniques. For instance, for a given sample, the complex analyte may be contacted with the binding composition under various conditions and the amount of antigen bound to the composition determined. This allows the definition of appropriate contacting conditions that do not saturate the binding capacity of the composition. Depending on the use, nature of the sample, etc., various conditions may be applied, ranging e.g., from very high to very low saturation conditions.

In this regard, in a particular embodiment, the contacting is performed under conditions allowing binding, to the binding composition, of up to 99% of all components of the complex analyte sample, so that 1% at most of the components of the complex analyte do not form antibody antigen complexes (“very high stringency conditions”).

According to an other embodiment, the contacting is performed under conditions allowing binding, to the binding composition, of between 95 and 99% of all components of the complex analyte sample, so that between 1-5% of the components of the complex analyte do not form antibody antigen complexes (“high stringency conditions”).

According to a further embodiment, the contacting is performed under conditions allowing binding, to the binding composition, of between 90 and 95% of all components of the complex analyte sample, so that between 5-10% of the components of the complex analyte do not form antibody antigen complexes (“medium stringency conditions”).

In another embodiment, the contacting is performed under conditions allowing binding, to the binding composition, of between 80 and 90% of all components of the complex analyte sample, so that between 10-20% of the components of the complex analyte do not form antibody antigen complexes (“low stringency conditions”).

Other conditions may be defined by the skilled person, without departing from the scope of the present invention, by adjusting the flow rate and/or analyte concentration in the sample.

As discussed above, the binding composition typically comprises a polyclonal antibody composition generated against the complex analyte sample. In particular embodiments, the binding composition comprises a derivative of the polyclonal antibody, e.g., a composition comprising substantially the same antigen repertoire as the polyclonal antibody. Such a derivative may comprise, for instance, a sub-fraction of the polyclonal, a dilution thereof, a depleted version thereof (e.g., wherein antibodies directed against particular antigens have been removed), a digestion product thereof, etc. The binding composition may comprise antibodies generated in any mammalian organism, particularly in non-human vertebrates, such as mammals: rodents (rabbits, mice, rats, etc.), horses, cows, goats, pigs, monkeys, camels, and birds: chickens, turkeys, etc.

Polyclonal antibodies against complex analyte samples may be produced by procedures generally known in the art. For example, polyclonal antibodies may be produced by injecting the complex analyte sample (or a derivative thereof), either alone or coupled to a suitable protein or potent adjuvant, into a non-human animal. After an appropriate period, the animal is bled, sera recovered and purified by techniques known in the art (see Paul, W. E. “Fundamental Immunology” Second Ed. Raven Press, NY, p. 176, 1989; Harlow et al “Antibodies: A laboratory Manual”, CSH Press, 1988; Ward et al (Nature 341 (1989) 544).

The binding composition may comprise a mixture of polyclonal antibodies having a different source and/or type.

In a particular embodiment, the binding composition comprises a polyclonal antibody obtainable by immunization of a non-human animal with a sample of human serum, human plasma, human bodily fluid, human tissue extract or environmental sample.

In a particular embodiment, the binding composition comprises immunoglobulins, derivatives thereof, serum or whole plasma comprising antibodies that react with a wide range of epitopes present in human, serum, human plasma, human bodily fluid, tissue extract or environmental sample.

In a further particular embodiment, the binding composition (or the polyclonal antibody) is conjugated to a solid matrix, under conditions that do not substantially alter antigen recognition and binding. Methods of immobilizing an antibody on a support are well known per se in the art. In a preferred embodiment, the solid matrix has a surface, coated with the polyclonal antibody.

In a further particular embodiment, the binding composition (the polyclonal antibody) is allowed to form complexes via Fc interactions with immobilized protein-G or protein-A, or other reagents that bind antibodies through the Fc portion, but do not change antigen binding capacity.

Within the context of this invention, a complex analyte sample designates a mixture of components, such as proteins, polypeptides, peptides and/or small molecules, whose composition is typically not precisely defined or known.

Examples of complex analyte samples include, for instance, (diluted) plasma, serum, urine or body fluid sample, a tissue extract or a cell lysate, of human, animal or plant origin; or an environmental sample. Examples of environmental samples include soil, water, cloud condensate, food processing intermediates and food products, cosmetics and other healthcare products. Other examples of complex analytes include any mixture that contains immunogen metabolites and/or immunogen proteins or peptides. The complex sample can also be a mix of individual and complex analyte samples, i.e., contain, in addition to a complex analyte mixture, known components. Immunogen metabolites include lipids, organic small molecules, sugars, complex sugars either in free state to bound to each other or to proteins or peptides (e.g. glycolipids, glycans, lipoproteins).

In a preferred embodiment, the complex analyte sample is a sample of or derived from human blood, plasma or serum.

In a typical embodiment, the method of this invention comprises the following steps:
1. Preparation of polyclonal antibody against a complex analyte via conventional immunization of mice, rats, rabbits, goats, horses or any other known vertebrate species that is known to act in response to immunization with a polyclonal antibody response;
2. Purification of immunoglobulins from sera of immunized vertebrates;
3. Conjugation of immunoglobulins (e.g. IgG, IgM or all Igs) to solid matrices, alternatively binding of immunoglobulins to immobilized Fc binding reagents like protein A or protein G;
4. Reacting the same or substantially similar (e.g. polyclonal anti human serum reacted with human serum from individuals whose serum was not used for the immunization) complex analyte mixture with immunoglobulins on the solid matrix at concentrations and conditions that do not saturate the antigen binding capacity of the conjugated immunoglobulins for the majority or any analyte class;
5. After the elimination of complex analytes that did not bind to the conjugated immunoglobulins the resulting mixture is a “normalized” sample relative to the original complex analyte mixture.

An alternative normalization strategy comprises:
6. Reacting the immunoglobulins and the complex analyte in solution;
7. Specifically separating: analyte elements that are complexed with immunoglobulins from those which are not. Mixture of analyte molecules that are not complexed represent a normalized mixture relative to the starting analyte mixture.

An other alternative normalization strategy comprises the generation of polyclonal antibody mix by mixing known number of monoclonal antibodies with the desired affinity and concentration and executing normalization steps as described above.

In a particular embodiment, the method further comprises a step of labelling the normalized analyte, e.g., with labels that provide physical and/or chemical signals in appropriate detection devices. A preferred example of label is biotin.

A further object of this invention resides in a normalized analyte sample obtainable by a method as disclosed above.

A further aspect of this invention resides in a method of producing a support for normalization of a complex analyte sample, the method comprising providing a binding composition comprising a polyclonal antibody generated against said complex analyte or a derivative thereof, and immobilizing said binding composition or a fraction thereof on a solid matrix. In a particular embodiment, the binding composition is depleted of particular antigen-binding antibody(ies).

A further object of this invention resides in a method of using normalized analyte samples of this invention, as an immunogen to generate complex mAb libraries. Typically, the normalized analyte sample is a normalized human serum, human plasma, human bodily fluid or environmental sample.

A further object of this invention relates to the use of a labelled normalized analyte of this invention:
in (a method of) direct capture immunoassays;
in microarray experiment where libraries of monoclonal capture antibodies are immobilized on the microarray surface. Detection of the antibody reactivity is possible with normalized complex analyte samples in ELISA capture assay. Quantitative measurement is possible by inhibition, using un-manipulated, depleted or normalized plasma as inhibitor, similarly to capture ELISA experiments;
for comparing physicochemical signals generated in mAb mediated capture assays;
for inhibiting binding of labelled normalized analyte samples by the addition of human plasma or serum to the immunoassays claimed under;
for inhibiting binding of labelled normalized analyte samples by the addition of depleted, fractionated human plasma or serum to the immunoassays claimed under;
for inhibiting binding of labelled normalized analyte samples by the addition of normalized human plasma or serum to the immunoassays claimed under;
for comparing inhibition due to human plasma samples derived from different individuals. In this respect, comparison is made between different individuals belonging to at least two groups, one having at least one disease condition. In a particular embodiment, where different individuals belong to at least two groups, one responding to a treatment by a drug.

Further aspects and advantages of the present invention will be disclosed in the following experimental section, which shall be regarded as illustrative and not limiting the scope of this application.

### Experimental Section

### LEGEND TO THE FIGURES

FIG. 1A. Biomarker discovery by antibody mediated proteomics process

FIG. 1B: Graphic interpretation summary of data mining analysis of MS results of samples normalized to various extents. (Black rectangles: no normalization, red circles: high stringency normalization, green triangles: medium stringency, blue triangles: low stringency). Reported plasma concentration of proteins is plotted against the number of peptides observed in the MS analysis

FIG. 2: A): Apparent relative analyte complexity of normalized serum protein samples as detected by MS technology (AG: depleted non normalized, H: high strignecy, M: medium stringency, L: low stringency normalization.) B): Apparent relative analyte complexity of normalized serum samples as detected by ELISA techniques (red: non normalized, blue: medium stringency, green: low stringency)

FIG. 3: Signal intensity distribution generated by biotinylated non normalized (Agilent) and normalized serum protein mixes (increments of 2-20 SDs, red line marks 2SD) Capture ELISA assay (GAM+mAB+biotinylated normalized plasma+ABC-PO)

FIG. 4: Titration of normal human plasma (pool 1 and pool2) into the tracer assay. Reaction of a hybridoma clones #270 and #24

FIG. 5: Monoclonal antibody library: reactivity with various tracers and with high abundant proteins

### MATERIALS AND METHODS

### Preparation of the Multi-ImmunoAffinity Normalization (MIAN) Column

The Anti-human whole serum (developed in rabbit, Sigma H3383, 23 mg/ml) was purified by adding 2 ml PBS to the antiserum vial and filtered on 22 μm spin filter (VivaScience, Palaiseau, France). The 1 ml HiTrap Protein G HP (Amersham Biosciences Europe GmbH, Orsay, France) column (Cat #17-0404-03) was equilibrated with PBS and the antiserum was applied by administering 3 injections of 600 μl filtered solution at 0.5 ml/min flow rate by means of an Amersham UPC-900 System (Amersham). Each step was followed by a washing step with PBS (unless mentioned otherwise). The washing was carried on until 280 nm absorbance of the flow through showed no apparent signal.

Cross linking of the protein G binding fraction of the Anti-human whole serum was accomplished by 5 consecutive injections of 2 ml of 150 mM DiMethyl Suberimidate.2HCl (DMS) and 150 mM DiMethyl Pimelimidate.2HCl (DMP) (Pierce Biotechnology, Perbio, Brebière, France) in 0.2M triethanolamin (pH8.4) with 0.5 ml/min flow rate. The column was washed with PBS between each injection at 0.5 ml/min flow rate. Then the 5×2 ml injections were repeated with a fresh DMS/DMP solution using 0.5 ml/min flow rate, followed by wash with PBS between each injection at 0.5 ml/min. Finally, 4 consecutive injections of 2.5 ml of 150 mM monoethanolamin (pH9.0) were applied at 1 ml/min flow rate to quench the remaining amine reactive groups. Once the cross linking process was finished, a “mock” elution step was included with 0.5 M Acetic acid, 1 M Urea, 100 mM NaCl (pH 2.8). This was followed by washing the column with PBS until the 280 nm absorbance signal of the detector reached zero again. In this step, a significant amount of protein leaves the column.

### Normalization Process

First, the 6 most abundant proteins were removed from the plasma samples by MARS technology (Agilent, Santa Clara, Calif.). The protein concentration of the resulting samples was adjusted to either 1 mg/ml or 1.7 mg/ml in PBS. This sample was then loaded onto the 1 ml bead volume Multi-ImmunoAffinity Normalization (MIAN) column by applying three different flow rates to accommodate the three required normalization stringencies. 0.2 ml/min loading speed was used for high stringency normalization, 0.5 ml/min loading speed was used for medium stringency normalization and 1.0 ml/min loading speed was used for low stringency normalization. After loading, a five minute equilibration/contact period was observed for each normalizations process. Then, the MIAN column was washed by PBS buffer with the same flow rates as during loading, i.e. 0.2 ml/min for high stringency, 0.5 ml/min for medium stringency and 1.0 ml/min for low stringency normalization, respectively. In all instances the initial loading flow-through and the wash flow through were combined, resulting in the differently normalized samples.

After the normalization process, the column bound proteins were eluted from the MIAN column by washing with an elution buffer containing 0.5 M Acetic acid, 1 M Urea, 100 mM NaCl (pH 2.8) at 1 ml/min flow rate until an OD280 nm=0. Protein composition of the eluate was tested by SDS-PAGE. The elution process was followed by the re-equilibration of the MIAN column with PBS buffer (at 1 ml/min for 5 min).

### Protein Identification Process

Proteins were identified using Mascot search engine with the composite, non-identical protein sequence database built from several primary source databases (MSDB-Matrix Sciences) restricted to the human sequences. In order to increase the confidence in the protein identification, two supplemental criteria were applied in addition to the Mowse probability score calculated in Mascot.
A protein hit has to include at least one unique peptide match to insure that duplicate or highly homologous proteins, are not included. Using Mascot, output this is achieved by insuring that at least one peptide is declared in bold letters or that a peptide in non-bold letters was not accepted in a different identification.
A protein has to include at least one peptide with an ion score higher than 25. By setting this threshold, we insure that at least one peptide match is not is not due to a random peptide identification.
When the protein annotation in MSDB was not sufficient to help identify clearly the protein, the sequence of the best peptide match was used to perform a BLAST search with the following parameters:
Matrix: PAM30
expect: 2000
word size: 2
complexity filter: not applied

### Results

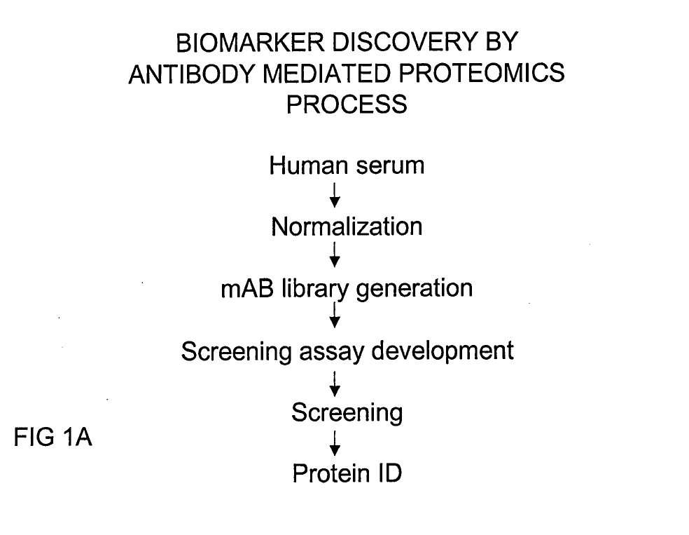

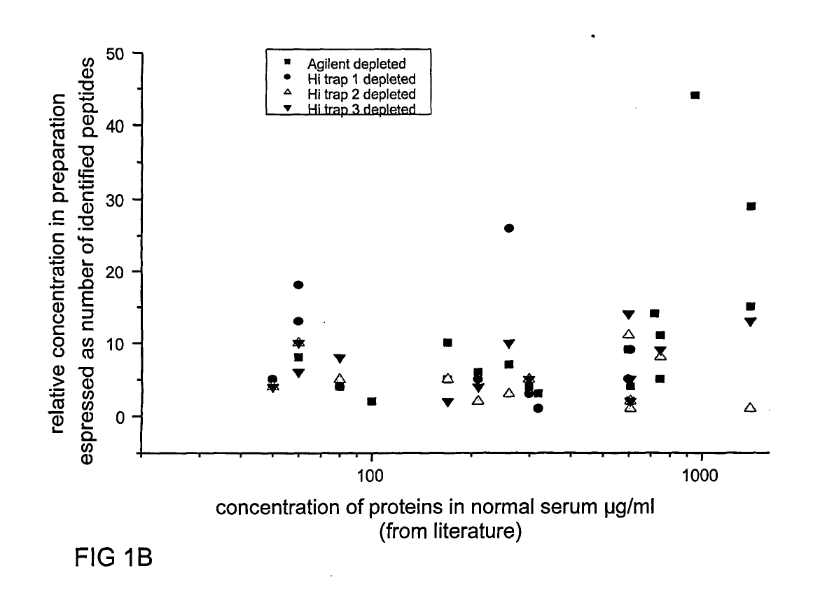

Anti-human serum was immobilized onto a separation column. Multiaffinity (Agilent) column depleted human serum samples were loaded on the column (FIG. 1A). The concentration of the loaded proteins and the loading speed were variable. For high stringency we used lower protein concentration and lower loading speed. Mass spectrometry analysis of the flow-through shows that each condition provided normalization to some extent, because the number of individual peptides derived from proteins that are present at higher concentration in the human plasma are represented similarly, while the non-normalized sample shows definitive correlation between the number of observed peptides and reported plasma concentration. Increasing the stringency of protein sample normalization reduces detection of proteins that are present at higher concentration in the plasma (FIG. 1B).

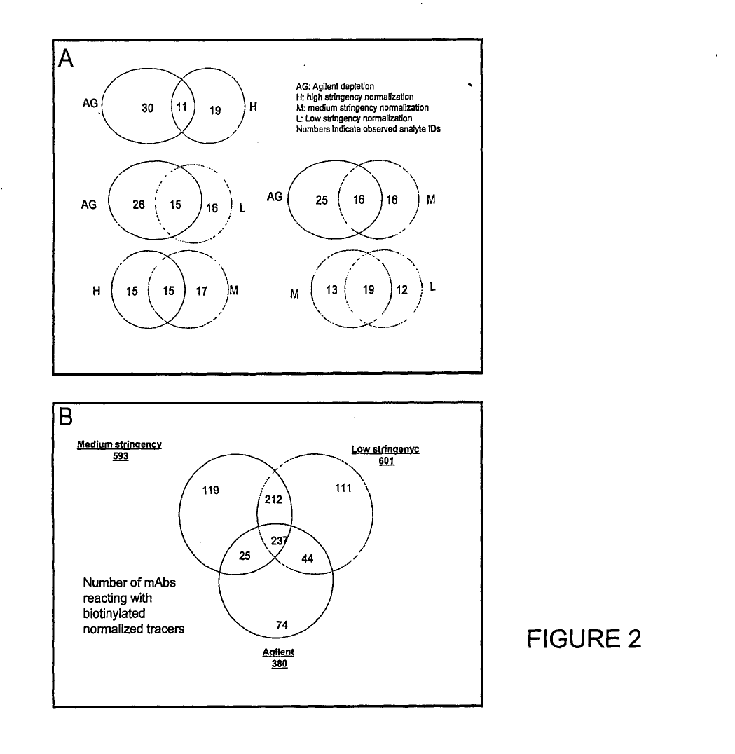

The composition of individual normalized protein samples was compared to each other by the results of MS based protein ID (FIG. 2 A) and ELISA tests (FIG. 2 B), using a large panel of monoclonal antibodies generated against complex human serum proteome samples. The results indicate that various normalization stringencies generated protein mixtures that contain overlapping but quite different protein elements. Normalized protein mixtures were biotinylated and applied as labelled tracers in ELISA experiments. The ELISA, plates were first coated with mouse Ig gamma-Fc specific GAM, then incubated with the mAb hybridoma supernatant, next the plates were incubated with biotinylated normalized protein mix, lastly ABC proxidase (Vector) was used to detect interaction between the mAb and the tracer.

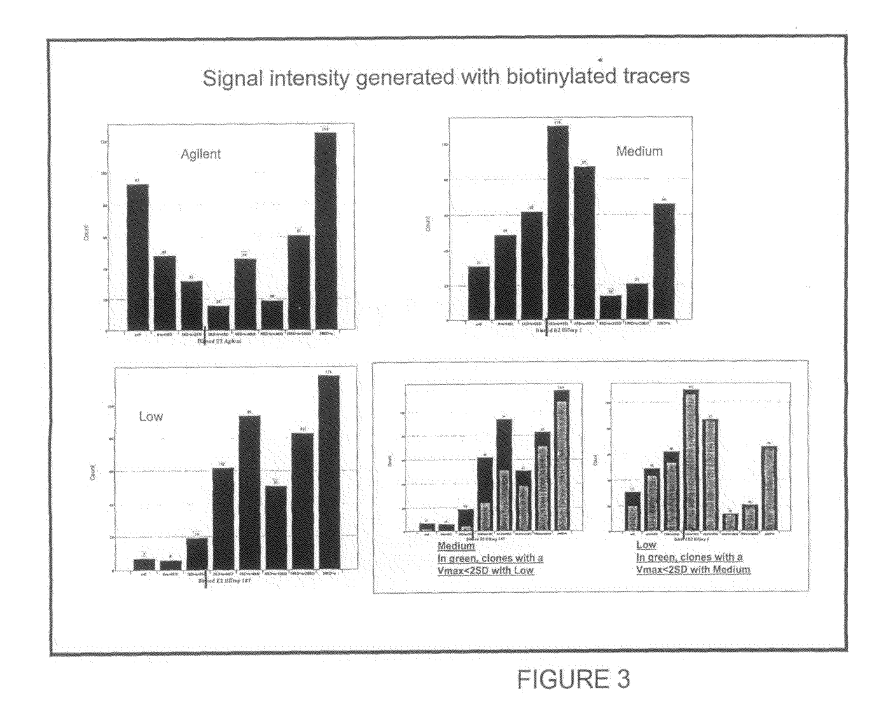

Signal intensity distribution shown in FIG. 3, indicates that the overall signal intensity is variable. Increasing the normalization stringency from low to medium level increases the signal of those (overlapping) clones that show relatively low signal with low stringency normalized tracer.

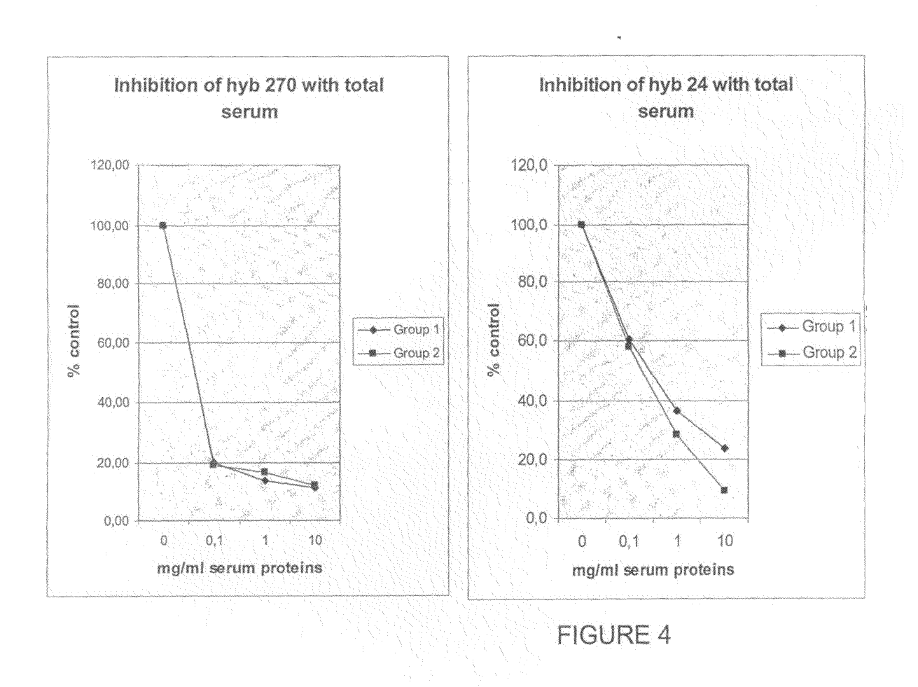

Signal generated by biotinylated tracer in the ELISA assay described above can be inhibited by normal human plasma. Titration of the plasma provides a quantification tool for relative but precise analyte-concentration measurement by the mAb-s that generate signal with a particular labeled tracer as depicted in FIG. 4.

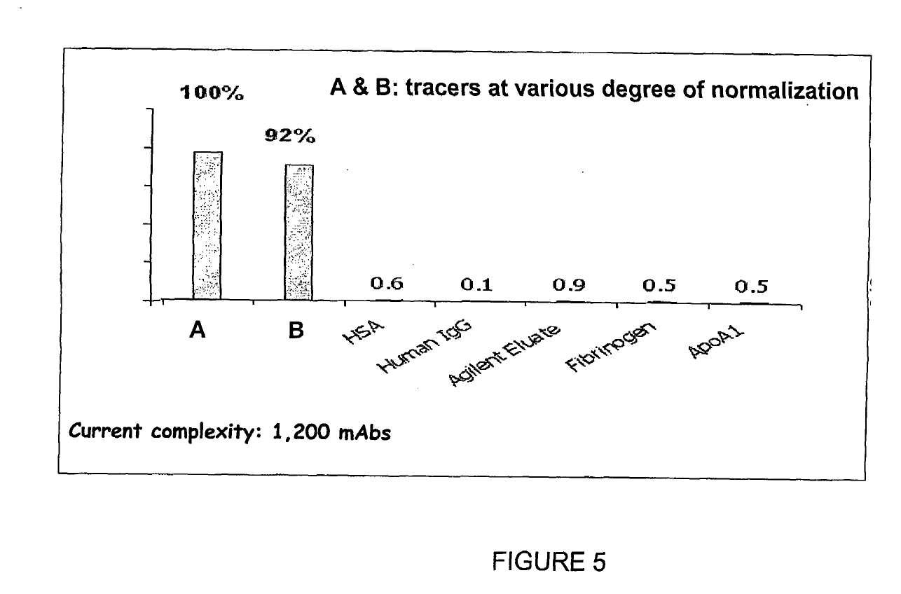

A monoclonal AB library (generated against human serum) reactivity with various tracers and with high abundant proteins is shown in FIG. 5.

1-31. (canceled)

32. A method of normalizing a complex analyte sample, the method comprising contacting said complex analyte sample with a binding composition comprising a polyclonal antibody generated against the complex analyte or a derivative thereof, under conditions that do not saturate the antigen-binding capacity of the binding composition, and recovering the sample that did not react with said binding composition, said sample being normalized.

33. The method of claim 32, wherein recovering the sample that did not react with the binding composition comprises removing all complexes formed.

34. The method of claim 32, wherein the contacting step is performed in solution.

35. The method of claim 32, wherein the binding composition is immobilized on a solid matrix.

36. The method of claim 35, wherein the solid matrix is selected from a bead, a stationary phase and a chromatography column.

37. The method of claim 32, wherein the conditions that do not saturate the antigen-binding capacity of the binding composition are achieved by limiting the interaction time between the sample and the binding composition.

38. The method of claim 37, wherein the binding composition is immobilized on a column and the interaction time is limited by changing the flow rate of the column.

39. The method of claim 32, wherein the conditions that do not saturate the antigen-binding capacity of the binding composition are achieved by limiting the analyte concentration in the sample.

40. The method of claim 32, wherein the contacting is performed under conditions allowing binding, to the binding composition, of up to 99% of all components of the complex analyte sample, so that 1% at most of the components of the complex analyte do not form antibody antigen complexes.

41. The method of claim 32, wherein the contacting is performed under conditions allowing binding, to the binding composition, of between 95 and 99% of all components of the complex analyte sample, so that between 1-5% of the components of the complex analyte do not form antibody antigen complexes.

42. The method of claim 32, wherein the contacting is performed under conditions allowing binding, to the binding composition, of between 90 and 95% of all components of the complex analyte sample, so that between 5-10% of the components of the complex analyte do not form antibody antigen complexes.

43. The method of claim 32, wherein the contacting is performed under conditions allowing binding, to the binding composition, of between 80 and 90% of all components of the complex analyte sample, so that between 10-20% of the components of the complex analyte do not form antibody antigen complexes.

44. The method of claim 32, wherein the derivative of the complex analyte sample used for polyclonal antibody generation comprises a sub-fraction of said analyte sample or a depleted analyte sample or a digestion product of the analyte sample.

45. The method of claim 32, wherein the polyclonal antibody is conjugated to a solid matrix.

46. The method of claim 45, wherein the solid matrix has a surface coated with the polyclonal antibody.

47. The method of claim 45, wherein the polyclonal antibody is immobilized on the solid matrix via Fc interactions with protein-G or protein-A immobilized on the solid matrix, or with any reagent that bind antibodies through the Fc portion.

48. The method of claim 32, wherein the complex analyte sample comprises a mixture of proteins, polypeptides, peptides and/or small molecules.

49. The method of claim 32, wherein the complex analyte sample is (diluted) plasma, serum, urine or body fluid sample, a tissue extract or a cell lysate, of human, animal or plant origin; or an environmental sample.

50. A method of producing a support for normalization of a complex analyte sample, the method comprising providing a binding composition comprising a polyclonal antibody generated against said complex analyte or a derivative thereof, and immobilizing said binding composition or a fraction thereof on a solid matrix.

51. The method of claim 50, wherein the binding composition is depleted of particular antigen-binding antibody(ies).

52. A method of using normalized analyte samples obtainable by a method of claim 32, as an immunogen to generate complex monoclonal antibody libraries.

53. A method according to claim 52, wherein the normalized analyte sample is a normalized human serum, human plasma, human bodily fluid or environmental sample.

54. A method of claim 32, further comprising a step of labelling the normalized analyte, e.g., with labels that provide physical and/or chemical signals in appropriate detection devices.

55. A method of claim 54, wherein the label is biotin.

56. A normalized analyte sample obtainable by a method of claim 32.

57. A labelled normalized analyte sample obtainable by a method of claim 54.

58. A direct capture immunoassay method, wherein the method comprises the use of a labelled normalized analyte of claim 26.

59. A method of comparing composition of complex analyte samples by mass spectrometry, the method comprising the use of a normalized analyte sample of claim 56.
